(19) United States
(12) Patent Application Publication
(10) Pub. No.: US 2008/0260724 A1
(43) Pub. Date: Oct. 23, 2008

(54) Drug Induced Exposure Of The Fungal Pro-Inflammatory Molecule Beta-Glucan

(75) Inventors: Robert T. Wheeler, Orono, ME (US); Gerald R. Fink, Chestnut Hill, MA (US)

Correspondence Address: PROSKAUER ROSE LLP, ONE INTERNATIONAL PLACE, BOSTON, MA, 02110, US

(73) Assignee: WHITEHEAD INSTITUTE FOR BIOMEDICAL RESEARCH, CAMBRIDGE, MA (US)

(21) Appl. No.: 12/048,559

(22) Filed: Mar. 14, 2008

Related U.S. Application Data
(63) Continuation of application No. PCT/US06/35812, filed on Sep. 14, 2006.
(60) Provisional application No. 60/716,895, filed on Sep. 14, 2005.

(30) Foreign Application Priority Data
Sep. 14, 2006 (US) PCT/US06/35812

(51) Int. Cl. A61K 39/395; C12Q 1/02; G01N 33/53; A61P 43/00; G01N 33/569

(52) U.S. Cl. 424/130.1; 435/29; 435/7.2; 435/7.31

(57) ABSTRACT

Methods and reagents for screening for anti-microbial agents are provided. Diagnostic tools for assessing treatment of fungal infections are provided. Antimicrobial substances including substances useful for the treatment of fungal infections are provided. In some embodiments, the substances have antigen unmasking activity. In some embodiments the substances have fungicidal activity and surface antigen unmasking activity.

### RELATED APPLICATIONS

This application is a continuation of PCT application PCT/US06/035812 filed Sep. 14, 2006, which claims the benefit of priority to U.S. provisional application 60/716,895, filed on Sep. 14, 2005; the entire teachings of both applications are incorporated herein in their entirety.

### GOVERNMENT SUPPORT

The invention described herein was supported in whole or in part by the National Institutes of Health Grant No. 11-1439-2202. The Government has certain rights in the invention.

### BACKGROUND

Microbial pathogens can be recognized by the immune system via surface antigens or via pathogen-associated molecular patterns (PAMPs), which can provoke pathogen-specific responses. PAMPs are recognized by pattern recognition receptors (PRRs), a class of proteins expressed by cells of the immune system. Recognition of microbial surface antigens or PAMPs by the immune system can facilitate the clearance or removal of the microbe from the host and can facilitate the destruction of the microbe.

Fungi are opportunistic pathogens responsible for occasionally severe disease in individuals with systemic disease. Candida albicans, a clinically important fungal pathogen, is recognized predominantly by two PAMPs, β-glucan and mannan, which account by weight for over 90% of its cell wall. The cell wall of these and other fungi is tiered, with an outer layer of mannoproteins covalently linked to an inner core of β-glucan. The inner β-glucan layer is an essential cell wall component targeted by fungicidal antibodies, immune receptors, and the echinocandin class of antifungal drugs. Anti-β-glucan antibodies can directly kill fungi and assist in the clearance of fungal infection. The β-glucan receptor Dectin-1 also recognizes fungi and mediates the innate immune system's pro-inflammatory response. The importance of this layer is also underscored by the effectiveness of the echinocandins, which bind to and inhibit β-glucan synthase causing cell lysis when used at high enough concentration (minimal inhibitory concentration).

Opportunistic fungal pathogens such as Candida albicans often cause fatal infections in patients with a compromised immune system. Unfortunately, current drugs often fail to halt fungal disease, are ineffective against drug-resistant strains, and have severe side effects. In addition, current clinical diagnosis and treatment of fungal infections often entails obtaining a patient sample and testing for the present of fungal cells bases on whether fungal cells can be cultured from the sample. Fungal cells that are cultured from the sample and antifungal agents are tested for their ability to kill the fungal cells in vitro. Typically, fungicides that do not kill the fungal cells are ruled out as possible treatments for that patient because it is deemed that the isolated fungal cells are resistant to the fungicide. There is a need for new compounds to fight microbial infections such as fungal infections as well as methods for screening compounds that may be useful to fight fungal infections.

### SUMMARY OF THE INVENTION

Diagnostic tools for fungal infections and methods of screening for anti-fungal agents are provided. As described herein, in nature, immunoreactive surface moieties such as antigens and/or PAMPs are not always accessible to the immune system of a host organism. In some instances, the surface antigen or PAMP is masked by other components of the cell wall or cell membrane of the microbe.

The underlying β-glucan in the cell wall of Candida albicans can be unmasked or exposed as provided herein. For example, sub-inhibitory doses of the antifungal drug caspofungin can be used to unmask β-glucan. Unmasking of β-glucan on the surface of fungal cells can lead to increased β-glucan receptor-dependent elicitation of key pro-inflammatory cytokines from macrophages. In addition, a genetic network involved in concealing or masking β-glucan from the immune system and limiting the host response is described. As described herein, this network is conserved among different fungal species. Perturbation of parts of this network in the pathogen C. albicans can cause unmasking of β-glucan, which can lead to increased β-glucan receptor-dependent elicitation of key pro-inflammatory cytokines from macrophages.

As a result of the unmasking of immunogenic surface moieties as described herein, microbial pathogens can be transformed into cells that elicit a strong pro-inflammatory response. For example, fungi such as C. albicans can be transformed into cells that elicit a strong pro-inflammatory response by unmasking β-glucan as described herein. In some embodiments, the compounds provided herein have dual action: ability to kill the pathogen and unmasking or exposure of one or more immunogenic surface moieties. In addition, as demonstrated herein, compounds that appear ineffective at inhibiting fungal cell growth in vitro may nevertheless be useful as an antimicrobial treatment or a component of antimicrobial treatment in vivo if such compounds cause the unmasking of microbial surface antigens such as β-glucan. Methods for accessing antifungal treatment are also provided herein.

In one aspect, methods for determining whether a compound enhances one or more immunogenic properties of a microbial cell are provided. In some embodiments, the method comprises contacting the compound with a sample comprising at least one microbial cell, and measuring an immunogenic property of the microbial cell, wherein an increase in the immunogenic property relative to an untreated microbial cell indicates that the compound enhances one more immunogenic properties of a microbial cell. In some embodiments, the compound contacts the microbial cell.

In other embodiments, methods for determining whether a compound has β-glucan exposing activity are provided. In some embodiments, the method comprises contacting the compound with a sample comprising at least one fungal cell that contains β-glucan, and determining whether β-glucan can be detected in the sample, wherein detection of the β-glucan indicates that the compound has anti-fungal activity. In some embodiments the compound contacts the fungal cell.

In another aspect, compounds identified by the methods are provided.

In still another aspect, methods of treating a patient by administering a compound identified by the methods described herein are provided.

In other embodiments, methods for determining whether a compound has anti-fungal activity and β-glucan exposing activity are provided. In some embodiments, the method comprises contacting the compound with a first sample comprising at least one fungal cell and determining whether the fungal cell of the sample has been inhibited, and contacting the compound with a second sample comprising at least one fungal cell and determining whether β-glucan can be detected in the sample, wherein inhibition of one or more fungal cells and detection of the β-glucan indicates that the compound has anti-fungal activity and β-glucan exposing activity. In some embodiments, the compound contacts the fungal cell.

In another aspect, methods for assessing antifungal treatment are provided. In some embodiments, the method for assessing antifungal treatment of a patient comprises measuring β-glucan exposure on fungal cells present in a sample isolated from the patient.

In other embodiments, the method for assessing potential antifungal treatment comprises contacting a first sample obtained from a patient with the candidate compound. The sample comprises at least one fungal cell that contains β-glucan. Whether the candidate compound inhibits the growth of the fungal cell in vitro is determined. A second sample obtained from a patient is contacted with a candidate compound. The second sample comprises at least one fungal cell that contains β-glucan. Whether β-glucan can be detected in the sample is determined. Detection of the β-glucan indicates that the compound is useful as an antifungal treatment.

In another aspect, kits for performing the methods described herein are provided. In some embodiments, a kit for determining whether a compound causes the exposure of β-glucan on a fungal cell is provided. In some embodiments, the kit comprises means for detecting β-glucan on fungal cells and means for measuring levels of pro-inflammatory response caused by the fungal cell.

The methods described herein are useful for screening compounds to identify potential anti-fungal compounds that can be used to treat fungal infection in a patient. In addition, as described herein, the methods can be used to determine whether a candidate compound is capable of enhancing the immunogenicity of a microbial cell. For example, as demonstrated herein, sub-inhibitory concentrations of antifungal agents can alter the fungal cell such that the fungal cell has enhanced immune properties. Enhanced immune properties, such as unmasking of surface antigens or PAMPs are expected to aid the host in clearing or eliminating the fungal infection from the body. Therefore, the present invention allows broader application of antimicrobial agents such as fungicides as well as the discovery of novel compounds having antifungal properties.

### BRIEF DESCRIPTION OF THE DRAWINGS

FIG. 1A shows TNFα production by RAW264.7 macrophages after exposure to candida strains WT(CAF2), kre5Δ/Δ, phr2Δ/Δ, WT(BWP17), ssn8Δ/Δ cells at a ratio of 2:1 candida to macrophage or by unexposed (null) macrophages.

FIG. 1B shows TNFα production by bone marrow-derived macrophages (BMDMs) after exposure to candida at ratios of 20:1, 5:1, 2:1 and 1:1 candida to macrophage.

FIG. 1C shows TNFα production by BMDMs after exposure to Candida strains WT(CAF2), kre5Δ/Δ, phr2Δ/Δ, WT(BWP17), ssn8Δ/Δ cells at a ratio of 10:1 candida to macrophage or by unexposed (null) macrophages.

FIG. 2A shows DIC (right panels) fluorescence (center panels) and overlay (left panels) of wild-type C. albicans treated with 0, ⅛, or ¼ MIC caspofungin and stained with anti-β-glucan antibody and Cy3 labeled secondary antibody.

FIG. 2B shows FACS analysis of wild-type C. albicans treated with 0, ⅛, or ¼ MIC caspofungin and stained with anti-β-glucan antibody and Cy3 labeled secondary antibody.

FIG. 2C shows the percent viability of wild-type C. albicans treated with 0 or ⅛, MIC of caspofungin.

FIG. 2D shows DIC (left), fluorescence (center) and overlay (right) images of wild-type C. albicans grown in RPMI and exposed (bottom panels) or not (top panels) to ¼ MIC of caspofungin.

FIG. 2E shows TNFα production by BMDMs exposed wild-type UV-inactivated C. albicans at a ratio of 10:1 yeast:macrophage ratio.

FIG. 3A shows β-glucan exposure on live, UV killed, or heat killed wild-type C. albicans as detected by anti-β-glucan antibody or Dectin-CRD.

FIG. 3B shows TNFα production by BMDMS exposed to UV killed or heat killed wild-type C. albicans at a ratio of 10:1 yeast to macrophage.

FIG. 4A shows bright field (top panels) and fluorescence (bottom panels) images of wild-type (right panels) or mutant (left panels, phr2Δ/Δ) C. albicans stained with anti-β-glucan antibody and labeled secondary antibody.

FIG. 4B shows the results from a different C. albicans mutant (kre5Δ/Δ).

FIG. 4C shows the results from a different C. albicans mutant (ssn8/Δ/Δ).

FIG. 4D-F show overlay histograms of FACS analysis and MFI data (insets) of the Phr2Δ/Δ), kre5Δ/Δ, and ssn8Δ/Δ mutants, respectively, each compared to wild-type.

FIG. 5 shows TNFα production by BMDMs exposed to different S. cerevisiae strains (wild-type, gas1Δ, or none) at a ratio of 5:1 yeast to macrophage.

FIG. 6 shows IL-6 production by BMDMs pretreated (light bars) with soluble β-glucan or not (dark bars) and exposed to different C. albicans strains (wild-type (CAF2), kre5Δ/Δ, kre5Δ/KRE5, phr2Δ/Δ, Phr2Δ/PHR2, wild-type (DAY286), ssn8Δ/Δ, or no fungi) at a ratio of 10:1 yeast to fungi.

### DETAILED DESCRIPTION OF THE INVENTION

In one aspect, methods for determining whether a compound enhances one or more immunogenic properties of a microbial cell are provided. In some embodiments, the method comprises contacting the compound with a sample comprising at least one microbial cell, and measuring an immunogenic property of the microbial cell, wherein an increase in the immunogenic property relative to an untreated microbial cell indicates that the compound enhances one more immunogenic properties of a microbial cell. In some embodiments, the compound contacts the surface of the microbial cell. In other embodiments, the compound diffuses into the microbial cell or is transported into the microbial cell. Where the cell has a cell wall and a cell membrane, such as a bacterial cell, for example, in some embodiments the compound enters the periplasmic space between the cell wall and the cell membrane.

In another aspect, compounds identified by the methods described herein are provided.

The various embodiments described herein can be complimentary and can be combined or used together in a manner understood by the skilled person in view of the teachings contained herein.

As used herein, microbial cell types include fungal cells, bacterial cells and protozoa. Fungal cells include human fungal pathogens. Human fungal pathogens include, for example, Candida sp such as C. albicans and Aspergillus sp such as A. fumigatus. Bacterial cells include human bacterial pathogens such as Borrelia burgdorferi, Staphylococcus aureus, Vibrio cholerae, and Clostridium botulinum. Protozoa include, for example Trypanosoma cruzi, Trypanosoma brucei, Leishmania spp., and Plasmodium falciparum.

As used herein, an immunogenic property includes the presence or exposure of one or more moieties on the surface of the cell. The immunogenic property (or enhancement thereof) can be measure, for example, by detecting binding of an immune factor to a surface of the microbial cell. The moiety can be, for example, a pathogen-associated molecular pattern (PAMP), an antigen, or other surface property that affects the ability of a first member of a binding pair to bind to a second member of the binding pair present on the surface of the microbial cell. Suitable PAMPs are described Wheeler, et al., PLoS Pathogens, 2(4):328-399 (2006).

The presence or exposure of one or more moieties on the surface of the microbial cell can be measured using, for example, a receptor, a lectin, or other molecule that is capable of binding the moiety. The presence or exposure of one or more moieties on the surface of the microbial cell can be measured using, for example, soluble immune factors, including antibodies (including IgG, IgA, IgM, and IgD) complement, mannose binding lectin (MBL), Pentraxins such as PTX3 and the like. Where the immunogenic property is the exposure or presence of an antigen or PAMP on the surface of the microbial cell, the immunogenic property can be measured by contacting the cell with antibody capable of binding the antigen. For example, where the microbial cell is a fungal cell, the microbial cell can be contacted with an anti-β-glucan antibody. Surface antigens or PAMPS for fungi, bacteria and protozoa are well known to those of ordinary skill in the art, and include, for example, β-glucan, mannan, bacterial lipopolysaccharide (LPS), lipoteichoic acid, and peptidoglycan. In another embodiment, measuring the immunogenic property can comprise contacting cell with receptor, such as an immune receptor, or portion of a receptor that is capable of binding the antigen or moiety. Suitable immune receptors include, for example, pattern recognition receptors such a β-glucan receptor. For example, where the microbial cell is a fungal cell, the microbial cell can be contacted with Dectin-1.

As used herein, an immunogenic property includes the ability of the microbial cell to elicit a pro-inflammatory response in vitro or in vivo. The ability of the microbial cell to elicit a pro-inflammatory response can be measured in vivo, for example by administering the microbial cells (after contacting the sample with the compound) to a host animal and measuring the ability a sample obtained from an infected host to elicit cytokine productions by macrophages in vitro.

The ability of the microbial cell to elicit a pro-inflammatory response can be measure in vitro, for example, by contacting immune cells with the microbial cells and measuring the level of cytokine production by the immune cells in response to exposure to the microbial cells. In some embodiments, the immune cells are primary macrophages. In some embodiments, a pro-inflammatory response is elicited when the microbial cells treated with the compound causes an increase in the level of pro-inflammatory cytokine production by immune cells compared to untreated microbial cells. Cytokine levels can be determined, for example, by collecting the medium in which the immune cells and the microbial cells were incubated together and performing Enzyme Linked Immunosorbant Assay (ELISA) using an antibody that is capable of binding the cytokine of interest.

In some embodiments the cytokine is a pro-inflammatory cytokine. Pro-inflammatory cytokines are cytokines that can stimulate the immune system to mount an immune response against the microbial cell and/or stimulates the immune system to clear the infection. Pro-inflammatory cytokines include, for example, TNFα and/or IL-6.

As used herein, one or more immunogenic properties of a microbial cell is considered enhanced, for example, if there is an increase in the property being measured compared to untreated microbial cells tested in the same assay. For example, where the immunogenic property includes the presence or exposure of one or more moieties on the surface of the cell, an enhancement includes the binding of more moiety-specific antibody or receptor compared to the binding of the moiety-specific antibody of receptor to untreated microbial cells, where binding is measured, for example by fluorescently labeled secondary antibody. Where the immunogenic property includes the ability of the microbial cell to elicit a pro-inflammatory response in vitro or in vivo, an enhancement includes, for example, the production of more cytokine by the treated microbial cells compared to untreated microbial cells, as measured, for example, by ELISA.

In some embodiments, the microbe can be inactivated or killed before the immune detection step. For example, some microbes are capable of killing primary macrophages in vitro, which could impair a pro-inflammatory response assay that uses primary macrophages. However, as demonstrated herein some fungal cells can become more reactive to β-glucan detection reagents, even when the β-glucan was masked in the live cell. Without wishing to be bound by theory, this may be due to the disruption of the microbial cell during the inactivation process, which causes the exposure of β-glucan. Therefore, in some embodiments, the method comprises inactivating the microbial cell such that the microbial cell remains intact. In an embodiment, the microbial cell is inactivated with ultraviolet light. In some embodiments, the microbial cell is a fungal cell and the fungal cell is inactivated with ultraviolet light. Microbial cells can be inactivated by exposing the microbial cell to one or more doses of at least 10,000 μjoules/cm². In some embodiments, additional doses are used to inactivate the microbial cells. In other embodiments a higher dose of inactivation is used. In an embodiment, four doses of at least 10,000 μjoules/cm² are used to inactivate fungal cells. In another embodiment, four doses of at least 100,000 μjoules/cm² are used to inactivate fungal cells. Where more than one dose is used, the sample of microbial cells can be agitated between each dose to treat cells evenly. In some embodiments, the microbial cells are washed and/or renormalized by optical density after UV inactivation.

The compound used in the methods provided herein can be a member of a library of natural products and/or synthetic (or semi-synthetic) products such as a small organic molecule library. These libraries can be prepared according to methods known in the art. Those skilled in the field of drug discovery and development will understand that the precise source of the compounds or libraries thereof is not critical to the methods described herein, including the screening methods. Accordingly, virtually any number of chemical compound or natural product library can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available for example, from QuantumLead on the World Wide Web at q-lead.com/cnt/home and Aldrich Chemical (Milwaukee, Wis.). Alternatively, libraries of natural compounds in the form of bacterial, fungal, plant, and animal extracts are commercially available from a number of sources.

The sample is contacted with suitable concentration of the compound. A range of concentration can be tested as described below. One of ordinary skill in the art can determine a suitable concentration or range of concentrations based on the microbe of interest and compound of interest using standard microbiological or clinical techniques. In some embodiments, the sample, (or second sample) is contacted with a sub-inhibitory concentration of the compound. As used herein, a sub-inhibitory concentration of a compound is a concentration at which, when exposed to a microbial cell in growth media, does not reduce the viability of the cell. Sub-inhibitory concentrations of a compound also include concentrations that do not alter the growth rate of the microbial cell. The range of sub-inhibitory concentrations for a given compound and a given microbial cell of interest can readily determined using standard microbiological techniques.

The compound can be a member of a library of compounds being screened for the ability to enhance one or more immunogenic properties of a microbial cell as described herein. The library can be pre-screened for particular properties. For example, the compound or library of compounds can be tested for the ability to inhibit certain genes or gene products of the microbial cell. Those compounds that inhibit the gene or gene products of interest can then be used in the methods described herein.

In some embodiments, the library or the compound is pre-screened for the ability to inhibit a gene or gene product involved in masking antigenic moieties or PAMPs associated with the surface of the microbial cell. The gene or gene product can be involved, for example, in the masking of β-glucan on the surface of a fungal cell. In some embodiments, the gene networks regulate cellular functions involved in polarized cell wall remodeling, polarization of the actin cytoskeleton, polarized secretion of proteins and polysaccharides, and/or polarized endocytosis of unwanted byproducts. In some embodiments, the compound inhibits the one or more genes or gene products involved in caspofungin sensitivity or caspofungin resistance. Genes involved in caspofungin sensitivity include SLT2, SLA1, and MNN10. In some embodiments, the compound inhibits genes or gene products involved in mannosylation of cell wall proteins. Genes involved in mannosylation of cell wall proteins include, for example, MNN10, MNN11, OCH1, OST3, and OST4.

Genes involved in masking of β-glucan on the surface of a fungal cell also include, for example, ACE2, ARC18, ARV1, ASF1, BEM1, BEM4, BNI1, CLA4, CNM67, CYK3, DIA2, EDE1, END3, ERD1, FAB1, FEN1, GAS1, GLO3, HEM14, HOF1, IES6, KRE28, KRE6, LAS21, PER1, RDS2, PRL16B, RVS161, RVS167, SAC3, SAC6, SEC66, MOT2, SSN8, TPD3, TUS1, VAC14, VRP1, YMR315W, YNL045W, YPL158C.

In addition, the compound or library of compounds can be pre-screened for the ability to inhibit homologs of the genes (or their gene products) described herein. As used herein, a homologous gene is, for example a gene having a sequence that is at least about 70% identical with a gene described herein. A homologous gene can also be at least about 80% or at least about 90% identical to a gene described herein.

Percent identity between two nucleic acid molecules can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), and BLASTP, BLASTN, FASTA (Atschul, S. F., et al., J Molec Biol 215:403 (1990)). For example, the BLAST function of the National Center for Biotechnology Information database can be used to determine identity. Other commercially or publicly available programs include, for example, DNAStar “MegAlign” program (Madison, Wis.) and the University of Wisconsin Genetics Computer Group (UWG) “Gap” program (Madison Wis.)).

In addition, the compound or library of compounds can be pre-screened for the ability to inhibit homologous genes (or their gene products) found in microbes other than S. cerevisiae. For example homologous genes (also referred to as orthologs) can be, for example, from Candida spp and Aspergillus spp.

In some embodiments, methods for determining whether a compound has β-glucan exposing activity are provided. In some embodiments, the method comprises the steps of contacting the compound with a sample comprising at least one fungal cell that contains β-glucan, and determining whether β-glucan can be detected in the sample, wherein detection of the β-glucan indicates that the compound has anti-fungal activity.

In some embodiments, methods for determining whether a compound has anti-fungal activity and β-glucan exposing activity are provided. In some embodiments, the methods comprise contacting the compound with a first sample comprising at least one fungal cell and determining whether the fungal cell of the sample has been inhibited, and contacting the compound with a second sample comprising at least one fungal cell and determining whether β-glucan can be detected in the sample, wherein inhibition of one or more fungal cells of a) and detection of the β-glucan indicates that the compound has anti-fungal activity and β-glucan exposing activity. As used herein, anti-fungal activity includes fungicidal activity and/or the ability to slow the growth rate of the fungal cells of interest.

In some embodiments, the compound can be tested for antibiotic or fungicidal activity. For example, in some embodiments, the method further comprises measuring viability of the microbial cell after contacting the compound with the sample comprising the microbial cell.

### Assessing Antimicrobial Treatment

In another aspect, methods for assessing antifungal treatment of a patient are provided. In some embodiments, the method comprises measuring β-glucan exposure on fungal cells present in a sample isolated from the patient. β-glucan exposure can be measured, for example, as described above, using a β-glucan receptor or an anti-β-glucan antibody, or by measuring the ability of the fungal cells of the sample to elicit a pro-inflammatory response in vitro.

In another aspect, methods for assessing potential antifungal treatment are provided. In some embodiments, the methods comprise contacting a first sample obtained from a patient with the candidate compound, the sample comprising at least one fungal cell that contains β-glucan and determining whether the candidate compound inhibits the fungal cell in vitro, and contacting a second sample obtained from a patient with a candidate compound, the sample comprising at least one fungal cell that contains β-glucan and determining whether β-glucan can be detected in the sample, wherein detection of the β-glucan indicates that the compound is useful as an antifungal treatment. As described herein, inhibition of microbial growth and β-glucan detection can be measured, for example, as described above. Inhibition of the fungal cell can include, for example, partial or complete (stasis) inhibition of growth, as well as loss of viability of the fungal cell.

Suitable patients include any animal, including humans that are susceptible to microbial and more specifically to fungal infection. Suitable first and or second samples as described herein can be obtained from a patient. Samples suitable for use in the present invention include any material suspected of containing a microbial cell. The sample can be any physiological fluid (e.g., blood, saliva, sputum, plasma, serum, ocular lens fluid, cerebrospinal fluid, sweat, urine, milk, ascites fluid, synovial fluid, peritoneal fluid, amniotic fluid, and the like). In addition, the sample can be biopsy material. The sample can be obtained from a human, primate, animal, avian or other suitable source. The patient sample can be used directly as obtained from the patient or following one or more steps to modify the sample. For example, plasma can be obtained from blood, viscous fluids can be diluted, and tissue samples can be broken apart.

Candidate compounds include, for example, compounds described above. Candidate compounds can include known antibiotics that fail to inhibit the microbial cells from the patient sample in vitro when used at or above the minimal inhibitory concentration. The candidate compound can be used at sub-inhibitory concentrations. The candidate compound can also include known antibiotics that inhibit microbial cells in vitro.

In another aspect, kits for determining whether a compound enhances one or more immunogenic properties of a microbial cell are provided. In some embodiments, the kits comprise a means for detecting β-glucan on fungal cells and a means for measuring levels of pro-inflammatory response caused by the fungal cell. Suitable means for detecting β-glucan on fungal cells include, for example, β-glucan receptor or portion thereof, and anti-β-glucan antibodies. Suitable means for measuring levels of pro-inflammatory response caused by the fungal cell include reagents for detecting cytokine production by immune cells. Such reagents can include, for example, primary macrophages, reagents for growing primary macrophages, and reagents for detecting and/or quantifying pro-inflammatory cytokines. In some embodiments, the kit includes anti-TNFα or anti-IL-6 antibodies.

The following examples are not intended to limit the present invention in any way.

### EXAMPLES

### Materials and Methods

Fungal Strains and Growth

S. cerevisiae strains in the BY4741 or BY4742 background were used to make the complete deletion library (Winzeler E A, et al. (1999) Science 285: 901). S. cerevisiae knockout libraries were purchased from Open Biosystems (Huntsville, Ala., United States). C. albicans strains were derived from clinical isolate SC5314 (Fonzi W A, Irwin MY (1993), Genetics 134: 717). Strain details are shown in Table 1.

TABLE I
Fungal strains

| Strain | | Strain | |
|---|---|---|---|
| Name | Genotype | Background | Species |
| BY4741 | MATa; his3Δ1; leu2Δ0; | S288c | S. cerevisiae |
| | met15Δ0; ura3Δ0 | | |
| BY4742 | matα his3Δ1; leu2Δ0; lys2Δ0; | S288c | S. cerevisiae |
| | ura3Δ0 | | |
| Deletions | BY4741 or BY4742 | S288c | S. cerevisiae |
| | orfX::kanMX6 | | |
| CAF2 | SC5314 ura3::imm434 | SC5314 | C. albicans |
| CFM2 | ura3::imm34/ura3::imm434 | CAF2 | C. albicans |
| | Δphr2::hisG/Δphr2::hisG− | | |
| | URA3-hisG | | |
| CFM3 | Δura3::imm434/Δura3::imm434 | CAF2 | C. albicans |
| | Δphr2::hisG/PHR2 | | |
| KAH3 | Δura3::imm434/Δura3::imm434 | CAF2 | C. albicans |
| | Δkre5::hisG/Δkre5::hisG− | | |
| | URA3-hisG | | |
| KAH4 | Δura3::imm434/Δura3::imm434 | CAF2 | C. albicans |
| | Δkre5::hisG/Δkre5::hisG + | | |
| | pLC14KRE5-URA3 | | |
| BWP17 | Δura3::imm434/Δura3::imm434 | SC5314 | C. albicans |
| | Δarg4::hisG/Δarg4::hisG | | |
| | Δhis1::hisG/Δhis1::hisG | | |
| ssn8Δ/Δ | Δura3::imm434/Δura3::imm434 | BWP17 | C. albicans |
| | Δarg4::hisG/Δarg4::hisG | | |
| | Δhis1::hisG/Δhis1::hisG | | |
| | Δssn8::URA3/Δssn8:ura3- | | |
| | ARG4-ura3 | | |

Fungi were grown overnight in YPD rich medium for yeast-form growth and RPMI 1640 for hyphal growth. C. albicans was grown at 37° C.; S. cerevisiae was grown at 30° C. For CF treatment, overnight cultures of C. albicans CAF2 were diluted 1:1,000 into fresh YPD or RPMI 1640 containing dilutions of CF (caspofungin acetate, Candidas formulation, Merck, Whitehouse Station, N.J., United States) and grown overnight. MIC50 was 2.5 ng/ml. For viability, cells were stained with propidium iodide (Sigma, St. Louis, Mo., United States).

Inactivation of Fungi for Macrophage Interaction Experiments

For UV inactivation, the equivalent of 2.5×10⁷ cells from a culture were washed and resuspended in 1 ml of PBS in a six-well plate. The fungi were exposed to four doses of 100,000 μjoules/cm² in a CL-1000 UV-crosslinker (UVP, Upland, Calif., United States), with agitation between each dose to treat cells evenly. For heat inactivation, 2.5×10⁷ cells in 1 ml of PBS were boiled for 10 min. After UV- or heat inactivation, cells were washed and renormalized by OD600. In S. cerevisiae, greater β-glucan exposure was found on cells that were heat-treated at 65° C. for 15 min, fixed for 30 min on ice in 3.7% formaldehyde, or fixed for 30 min on ice in 70% ethanol.

Screen for β-Glucan Exposure.

Overnight cultures of strains were grown in YPD and stained with anti-β-glucan primary antibody (Biosupplies Inc., Parkville, Australia) and Cy3-labeled goat-anti-mouse secondary. This antibody is specific for β1,3-glucan (Meikle P J, et al., (1991), Planta 185:1), and costaining with this antibody and the purified Dectin-CRD shows colocalization. Stained cells were adhered to clear-bottom plates with concanavalin A (Sigma) and scanned with Cellomics VTI fluorescence microscopic imager (Cellomics, Pittsburgh, Pa., United States) using Target Acquisition software (Zeiss, Oberkochen, Germany). Mean average intensity and standard deviation of average intensity measurements were used to identify strains with over two standard deviations greater β-glucan exposure. Initially matA mutant strains were screened, then the independently constructed matα counterparts were screened. Dectin-CRD was expressed from pTrcHis2 vector (Invitrogen, Carlsbad, Calif., United States), purified from E. coli as described (Gantner B N, et al., (2005), EMBO J. 24:1277), and labeled directly with Alexa Fluor 555-succinimidyl ester (Molecular Probes, Eugene, Oreg., United States). Fungi were labeled with pure protein and showed glucan-inhibitable binding and similar staining pattern to that described.

Macrophages

RAW264.7 macrophages (ATCC, Manassas, Va., United States) were cultured in RPMI-10 (RPMI 1640 with 10% heat-inactivated fetal bovine serum and standard concentrations of penicillin-streptomycin; GIBCO, San Diego, Calif., United States). Cells were collected and plated in 24-well plates at 5×10⁵ cells/well. Bone marrow derived macro-phages (BMDMs) were differentiated as described (Brown G D, et al., (2003), J Exp Med 197:1119). Briefly, bone marrow from BL/6 mice was cultured in RPMI-10 with 15% L-cell conditioned media and 25 ng/ml recombinant M-CSF (R&D Systems, Minneapolis, Minn., United States). Cells were collected after 6 d and plated in 24-well plates at 5×10⁵ cells/well.

Macrophage-Yeast Interaction

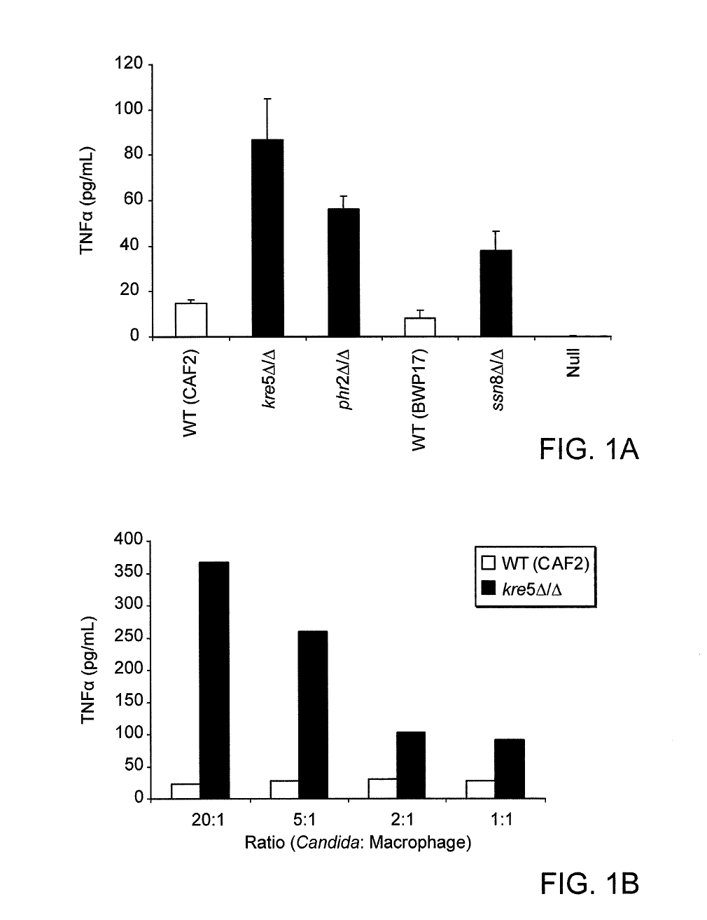

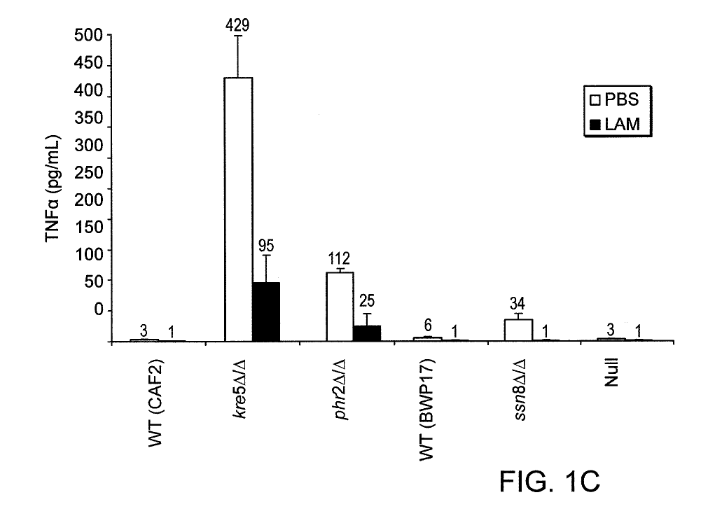

Fungi were grown overnight, washed extensively to remove any shed proteins or polysaccharides, and normalized by OD600 or hemacytometer (these two methods produced equivalent results). For S. cerevisiae, live fungi were added to RAW264.7 macrophages at equivalent effector:target (E:T) ratio of 1:5 in 24-well plates and incubated for 6 h, when supernatants were collected for ELISA. For exposure of C. albicans, UV-inactivated fungi were exposed either to RAW264.7 macrophages at an equivalent E:T of 1:2 or to BMDMs at an E:T of 1:10. For RAW264.7 exposures, supernatants were collected after 2 h; for BMDM exposures, supernatants were collected after 6 h. For blocking, BMDMs were preincubated with 100 μg/ml laminarin for 20 min on ice, exposed to fungi for 30 min at 37° C., washed extensively, then incubated for 6 h more at 37° C. ELISAs were performed according to the manufacturer's instructions (R&D Systems). Note that soluble β-glucan (laminarin) alone does not stimulate the macrophages (FIG. 1C).

Staining, Microscopy, And FACS

Fungi were grown overnight, washed, blocked in PBS+2% BSA, and stained with either the anti-β-glucan antibody (followed by Cy3- or PE-labeled secondary antibody), Alexa Fluor-labeled Dectin-CRD, or Dectin-CRD-anti-Myc. Staining with antibody and Dectin-CRD is described above for the screen. For staining with Dectin-CRD-anti-Myc, Dectin-CRD was preincubated with FITC-labeled anti-Myc (Invitrogen) at a ratio of 3.4 μg of Dectin-CRD+10 μl of anti-Myc, and then added to fungi at 1:100 on ice. Preincubation of Dectin-CRD with anti-Myc creates a probe with the same approximate size as the anti-β-glucan antibody but with the specificity of Dectin-1. Images of stained cells were taken using a Nikon TE2000-S microscope (Nikon, Tokyo, Japan) equipped with Spot RT camera (Diagnostic Instruments, Sterling Heights, Mich., United States) and processed in Photoshop (Adobe Systems, Palo Alto, Calif., United States). Fluorescence was quantified on a FACScalibur cytometer (Becton-Dickinson, Palo Alto, Calif., United States); cells were gated by forward and side scatter based on wild-type cell size and shape, and mean fluorescence intensity of 20,000 labeled cells was calculated using Cellquest software (Becton-Dickinson).

### Fungal β-Glucan is Exposed by Sub-Inhibitory Doses of Caspofungin

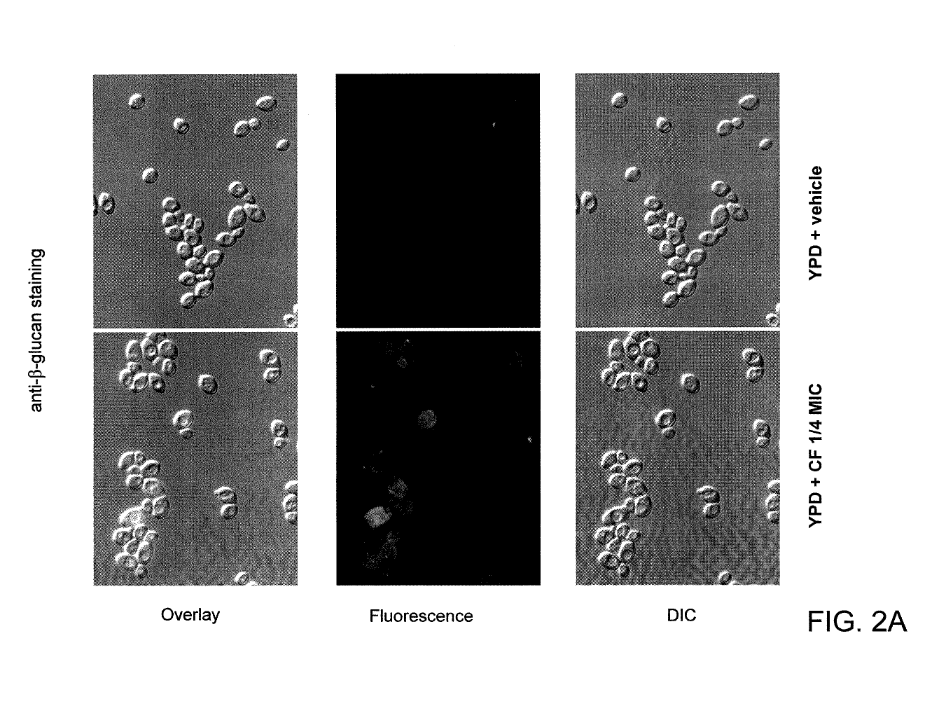

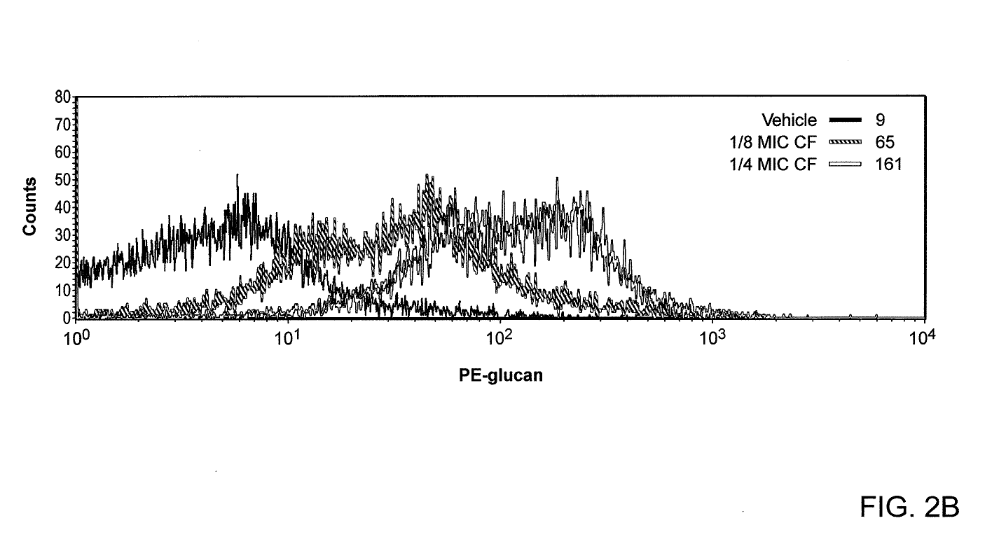

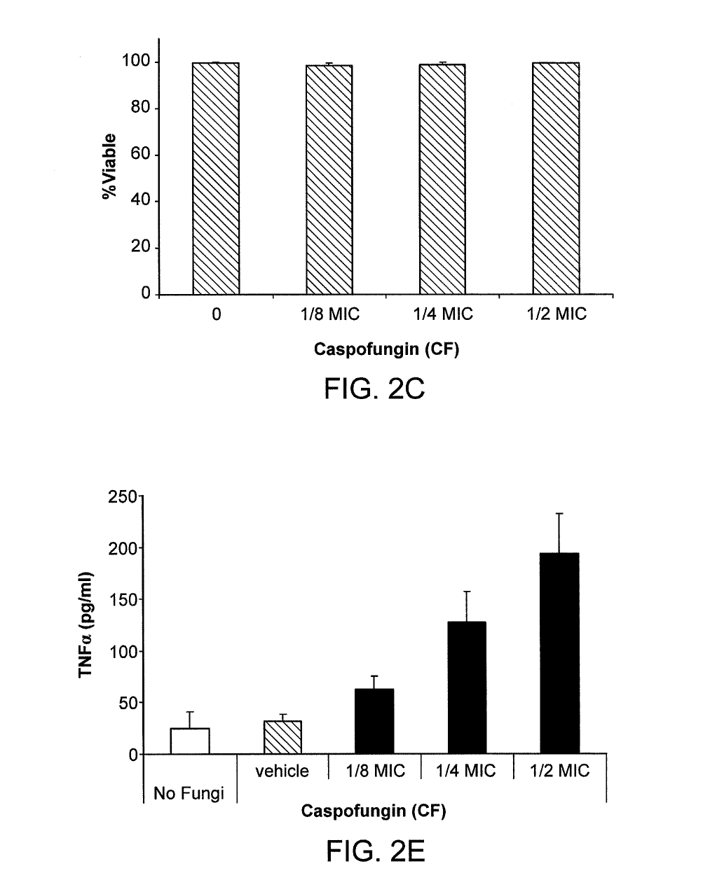

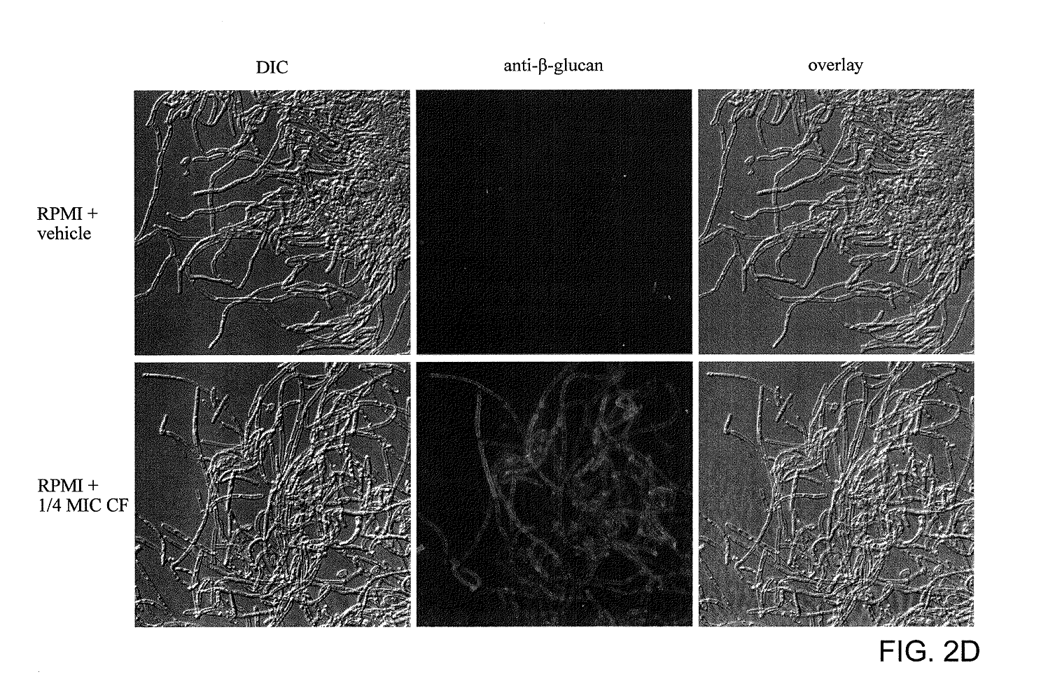

Wild-type C. albicans were grown overnight in the presence of sub-inhibitory doses of the echinocandin, caspofungin (CF). Remarkably, at CF concentrations that permit normal growth rates the drug caused exposure of β-glucan on the C. albicans cell surface (FIGS. 2A and 2B). At these CF concentrations, the cells had 10×-30× greater reactivity with the anti-β-glucan antibody. It is important to note that the fungi grow at these drug concentrations without loss of viability (FIG. 2C), suggesting that the increased β-glucan exposure is not due to generalized cell death. The effect of CF on cells grown in RPMI 1640, a culture medium that causes strong hyphal growth and β-glucan masking was also tested. Subinhibitory doses of CF caused a dramatic increase in the exposure of β-glucan on hyphae grown in this media (FIG. 2D). These cells also did not show increased cell death, as measured by microscopic examination.

FIG. 2 shows wild-type C. albicans (CAF2) grown overnight for ten generations in YPD medium, favoring yeast-form growth (FIGS. 2A, B, C, and E) or RPMI medium, favoring hyphal growth (FIG. 2D). Cultures grown at one-quarter and one-eighth of the CF MIC50 (minimal inhibitory concentration to inhibit 50% of the organisms) were stained with anti-β-glucan antibody and Cy3-labeled secondary antibody (FIGS. 2A and D) for visualization by microscopy or with PE-labeled secondary antibody (FIG. 2B) for FACS quantification. Mean fluorescence intensity (MFI) values were 9, 65, and 161, respectively, for no treatment, one-eighth the CF MIC50, and one-quarter the CF MIC50. Concurrently, cells were labeled briefly with propidium iodide to assess viability and visualized by epifluorescence microscopy or quantified by FACS (FIG. 2C). Cells grown overnight in YPD with or without CF were UV-inactivated and then exposed to BMDMs at a yeast:macrophage ratio of 10:1. Supernatants were taken at 6 h and assayed for TNFα (FIG. 2E).

### Caspofungin-Treated Cells Hyperelicit TNFα

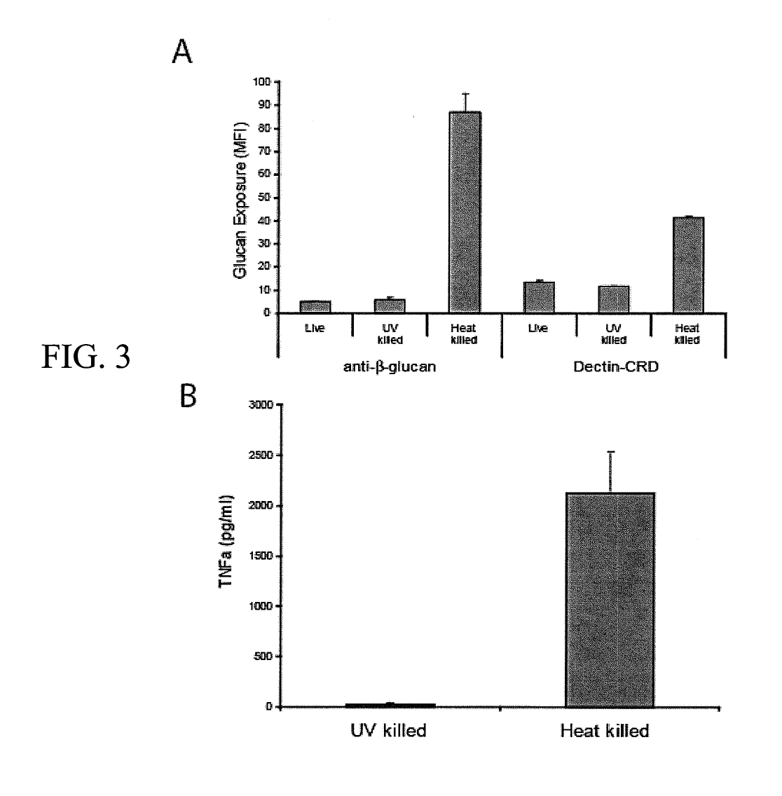

The ability of yeast unmasked by CF to elicit larger pro-inflammatory responses from macrophages was tested. In some embodiments, fungi were inactivated to prevent the fungi from killing the macrophages before the end of the experiment. Previously used methods of inactivation (including heat, formaldehyde, and ethanol) artificially increase β-glucan exposure and lead to high levels of TNFα elicitation (FIG. 3). It was found that ultraviolet light (UV)-irradiated cells did not kill macrophages, retained an intact cell wall architecture, and elicited only low levels of TNFα (FIG. 3). Therefore, UV-inactivated fungi were used for all subsequent experiments that required inactivation of the fungi. S. cerevisiae do not typically kill macrophages and therefore do not necessarily require inactivation.

FIG. 3 shows wild-type (CAF2) fungi grown overnight in YPD medium at 37° C. Cells were killed by UV irradiation or by heat inactivation (10 min at 100° C.). In FIG. 3A, live or killed cells were probed with anti-β-glucan antibody and PE-labeled secondary antibody and with Alexa Fluor 488-labeled Dectin-CRD, then subjected to FACS analysis. In FIG. 3B, UV- or heat-killed cells were then exposed to BMDMs at a ratio of 10:1 (yeast:macrophage), and supernatants were taken after 6 h for measurement of TNFα levels.

To test whether unmasked, CF-treated yeast elicit a greater pro-inflammatory response, CF-treated or -untreated (exposed or masked, respectively) C. albicans were exposed to BMDMs and assayed elicitation of the key pro-inflammatory cytokine TNFα. The CF-treated C. albicans elicited 3- to 4-fold higher levels of the key pro-inflammatory cytokine TNFα (FIG. 2E) than did untreated C. albicans, which elicited undetectable levels of TNFα in this assay. Thus, sub-inhibitory doses of CF cause the pathogen to elicit a marked pro-inflammatory response.

### An Interconnected Genetic Network is Required for β-Glucan Masking

To identify the system used by fungi to mask β-glucan from the immune system, a genome-wide library of knockout mutants in S. cerevisiae was screened for increased β-glucan exposure. Because sub-inhibitory levels of CF are able to cause increased β-glucan exposure and altered recognition of the fungi without killing the fungi, this library of nonessential gene knockouts should identify genes that specifically perturb the genetic network required for β-glucan masking. Further, due to similarity in cell wall structure between these two fungi, the genes identified in Saccharomyces lead to functionally equivalent genes in Candida that may serve the same masking function.

Using automated microscopy, the entire library of approximately 4,800 S. cerevisiae mutants was screened for strains with increased β-glucan exposure. This platform permits quantification of the fluorescence from high resolution pictures of the β-glucan-stained yeast. This method of quantification is exemplified in the comparison of the vrp1Δ mutant to wild-type. Strains with levels of antibody binding greater than two standard deviations from the mean of each plate were identified, and independently created mutants were re-screened using the same protocol. The genome-wide set of S. cerevisiae knockout mutants was screened by staining with anti-β-glucan antibody and quantifying β-glucan exposure using the Cellomics system. Object-finding software used the ConA fluorescence signal to identify cells and quantify the average level of fluorescence from the anti-β-glucan channel for each cell. Wild-type yeast showed little or no fluorescence from the anti-β-glucan channel, whereas the unmasked vrp1Δ mutant showed high levels of fluorescence from this channel.

Most of the mutants found with increased β-glucan exposure also showed greater binding to Dectin-CRD and increased TNFα elicitation from RAW 264.7 macrophages. Of the 76 mutants identified with increased anti-β-glucan binding, 48 hyperelicited TNFα from macrophages and 65 showed increased binding to the labeled Dectin-CRD. Forty-four mutants identified had increased anti-β-glucan binding and increased Dectin-CRD binding and increased TNFα elicitation from macrophages.

Emphasizing the connection between β-glucan exposure and immune recognition, most of the mutants with greater exposed β-glucan also showed increased binding to the Dectin-1 β-glucan receptor. Each S. cerevisiae mutant was exposed to RAW264.7 murine macrophages and tested for TNFα elicitation. A large percentage (48 of 76) of the unmasked mutants triggered a significantly stronger pro-inflammatory response than did the wild-type laboratory strain of S. cerevisiae; some elicited up to ten times the amount of TNFα as did wild-type (Table II).

TABLE II
Overall phenotype of hyper-eliciting mutants

| YORF | | | ANTI-β- | DECTIN- | | TNFα |
|---|---|---|---|---|---|---|
| NUMBER | NAME | Proteome Database Description | GLUCAN | CRD | TNFα | (% WT) |
| YLR131C | ACE2 | Metallothionein expression | 2 | 2 | 3 | 499 |
| | | activator with similarity to Swi5p, | | | | |
| | | has three tandem C2H2-type zinc | | | | |
| | | fingers, required for delaying G1 | | | | |
| | | phase specifically in daughter | | | | |
| | | cells | | | | |
| YLR370C | ARC18 | Component of the ARP2/3 actin- | 2 | 3 | 3 | 447 |
| | | organizing complex, involved in | | | | |
| | | actin assembly and function | | | | |
| YLR242C | ARV1 | Protein involved in sterol uptake | 2 | 1 | 1 | 164 |
| | | and distribution into the plasma | | | | |
| | | membrane, required for normal | | | | |
| | | sphingolipid metabolism | | | | |
| YJL115W | ASF1 | Anti-silencing function 1, a | 1 | 2 | 1 | 207 |
| | | component of replication-coupling | | | | |
| | | chromatin assembly factor | | | | |
| | | (RCAF) | | | | |
| YBR200W | BEM1 | Protein required for cell | 1 | 1 | 2 | 360 |
| | | polarization and bud formation | | | | |
| YPL161C | BEM4 | Bud emergence protein that | 1 | 0 | 1 | 227 |
| | | interacts with Rho-type GTPases | | | | |
| YNL271C | BNI1 | Stimulates actin (Act1p) filament | 1 | 1 | 2 | 286 |
| | | assembly and protects growing | | | | |
| | | actin ends from excess capping | | | | |
| | | protein, required for bipolar | | | | |
| | | budding pattern | | | | |
| YNL298W | CLA4 | Serine/threonine protein kinase | 1 | 2 | 2 | 321 |
| | | required for cytokinesis | | | | |
| YNL225C | CNM67 | Protein involved in nuclear | 2 | 3 | 3 | 734 |
| | | migration and component of the | | | | |
| | | spindle pole body | | | | |
| YDL117W | CYK3 | Protein involved in cytokinesis | 1 | 2 | 1 | 247 |
| YOR080W | DIA2 | Protein involved in invasive | 2 | 1 | 2 | 280 |
| | | growth, contains an F-box, | | | | |
| | | tetratricopeptide (TPR) repeats | | | | |
| | | and leucine-rich (LRR) repeats | | | | |
| YBL047C | EDE1 | Protein with role in endocytosis | 2 | 2 | 3 | 421 |
| YNL084C | END3 | Protein required for endocytosis | 2 | 2 | 3 | 634 |
| | | and cytoskeletal organization | | | | |
| YDR414C | ERD1 | Protein required for retention of | 2 | 1 | 2 | 301 |
| | | luminal ER proteins | | | | |
| YFR019W | FAB1 | Phosphatidylinositol-3-phosphate | 1 | 3 | 1 | 205 |
| | | 5-kinase involved in orientation or | | | | |
| | | separation of mitotic | | | | |
| | | chromosomes | | | | |
| YCR034W | FEN1 | Protein involved in the elongation | 2 | 2 | 1 | 217 |
| | | of fatty acids up to 24 carbons | | | | |
| YMR307W | GAS1 | 1,3-beta-Glucanosyltransferase, | 2 | 1 | 2 | 281 |
| | | glycophospholipid-anchored | | | | |
| | | surface glycoprotein that | | | | |
| | | regulates the crosslinking of beta- | | | | |
| | | 1,6-glucans in the cell wall | | | | |
| YER122C | GLO3 | GTPase-activating protein (GAP) | 2 | 0 | 2 | 293 |
| | | for ADP-ribosylation factors Arf1p | | | | |
| | | and Arf2p, involved in retrograde | | | | |
| | | transport between Golgi and ER | | | | |
| | | as well as endocytosis | | | | |
| YER014W | HEM14 | Protoporphyrinogen oxidase, | 2 | 1 | 2 | 198 |
| | | converts protoporphyrinogen to | | | | |
| | | protoporphyrin during heme | | | | |
| | | biosynthesis | | | | |
| YMR032W | HOF1 | Homolog of cdc15 (S. pombe | 1 | 2 | 3 | 627 |
| | | cdc15) 1, an SH3 domain- | | | | |
| | | containing protein involved in | | | | |
| | | cytokinesis, required for formation | | | | |
| | | of a novel actin belt structure in | | | | |
| | | the bud neck | | | | |
| YEL044W | IES6 | Ino Eighty Subunit 6 | 1 | 2 | 1 | 212 |
| YDR532C | KRE28 | Protein of the spindle pole body, | 1 | 1 | 2 | 365 |
| | | forms a complex with Spc105p | | | | |
| YPR159W | KRE6 | Glucan synthase subunit required | 3 | 2 | 3 | 997 |
| | | for synthesis of beta-1,6-glucan | | | | |
| YJL062W | LAS21 | Protein required for addition of a | 2 | 0 | 1 | 172 |
| | | side chain to the | | | | |
| | | glycosylphosphatidylinositol (GPI) | | | | |
| | | core structure | | | | |
| YDR245W | MNN10 | Subunit of the M-Pol II | 3 | 2 | 3 | 1024 |
| | | mannosyltransferase complex | | | | |
| YJL183W | MNN11 | Subunit of the M-Pol II | 3 | 2 | 3 | 1080 |
| | | mannosyltransferase complex | | | | |
| YGL038C | OCH1 | Alpha-1,6-mannosyltransferase | 2 | 2 | 3 | 983 |
| YOR085W | OST3 | Oligosaccharyltransferase | 2 | 0 | 1 | 156 |
| | | gamma subunit | | | | |
| YDL232W | OST4 | Oligosaccharyltransferase subunit | 3 | 1 | 3 | 425 |
| YCR044C | PER1 | Molecular function unknown | 3 | 1 | 1 | 201 |
| YPL133C | RDS2 | Protein with similarity to | 1 | 1 | 1 | 140 |
| | | transcription factors | | | | |
| YNL069C | RPL16B | Ribosomal protein L16 | 2 | 2 | 1 | 174 |
| YCR009C | RVS161 | Protein required for viability after | 3 | 3 | 2 | 312 |
| | | nitrogen, carbon, or sulfur | | | | |
| | | starvation, also required for | | | | |
| | | internalization step of endocytosis | | | | |
| | | and for cell fusion during mating | | | | |
| YDR388W | RVS167 | Protein that affects actin | 3 | 3 | 2 | 383 |
| | | distribution and bipolar budding | | | | |
| YDR159W | SAC3 | Nuclear protein required for | 2 | 3 | 2 | 396 |
| | | mRNA export from the nucleus to | | | | |
| | | the cytoplasm and for leucine | | | | |
| | | transport, mutants exhibit defects | | | | |
| | | in cytoskeletal function and in | | | | |
| | | mitosis | | | | |
| YDR129C | SAC6 | Fimbrin, an actin filament | 3 | 3 | 1 | 243 |
| | | bundling protein essential for | | | | |
| | | polarized secretion | | | | |
| YBR171W | SEC66 | Component of ER protein- | 1 | 1 | 3 | 458 |
| | | translocation subcomplex with | | | | |
| | | Sec62p, Sec63p, Sec66p, and | | | | |
| | | Sec72p | | | | |
| YER068W | MOT2 | Zinc finger transcriptional | 2 | 3 | 2 | 273 |
| | | repressor, involved in G protein | | | | |
| | | mediated pheromone signal | | | | |
| | | transduction and member of the | | | | |
| | | CCR4-Not complex | | | | |
| YBL007C | SLA1 | Protein involved in assembly of | 3 | 3 | 3 | 676 |
| | | cortical actin cytoskeleton, has | | | | |
| | | three SH3 domains | | | | |
| YHR030C | SLT2 | Serine-threonine protein kinase of | 1 | 1 | 2 | 369 |
| | | the MAP kinase family involved in | | | | |
| | | the cell wall integrity (low- | | | | |
| | | osmolarity) pathway and in G2 | | | | |
| | | phase cell-cycle checkpoint | | | | |
| | | control | | | | |
| YNL025C | SSN8 | Cyclin C homolog, component of | 1 | 2 | 1 | 246 |
| | | RNA polymerase holoenzyme | | | | |
| | | complex and Kornberg's mediator | | | | |
| | | (SRB) subcomplex | | | | |
| YAL016W | TPD3 | Regulatory A subunit of protein | 1 | 2 | 3 | 607 |
| | | serine-threonine phosphatase 2A, | | | | |
| | | activated by ceramide, required | | | | |
| | | for normal shmoo formation and | | | | |
| | | mating efficiency | | | | |
| YLR425W | TUS1 | GDP-GTP exchange factor for | 2 | 3 | 1 | 249 |
| | | Rho1p, involved in the cell | | | | |
| | | integrity signaling pathway | | | | |
| YLR386W | VAC14 | Protein involved in Fab1p- | 2 | 3 | 2 | 290 |
| | | dependent | | | | |
| | | phosphatidylinositol(3,5) | | | | |
| | | bisphosphate synthesis | | | | |
| YLR337C | VRP1 | Proline-rich protein verprolin, | 3 | 3 | 3 | 1048 |
| | | involved in cytoskeletal | | | | |
| | | organization and cellular growth | | | | |
| YMR315W | YMR315W | Member of the oxidoreductase | 1 | 1 | 1 | 160 |
| | | family NAD-binding Rossmann | | | | |
| | | fold family, contains a GFO, IDH, | | | | |
| | | or MOCA oxidoreductase C- | | | | |
| | | terminal alpha or beta domain | | | | |
| YNL045W | YNL045W | Bifunctional leukotriene A4 | 1 | 0 | 1 | 157 |
| | | hydrolase and anion-activated | | | | |
| | | leucyl aminopeptidase | | | | |
| YPL158C | YPL158C | Protein of unknown function | 2 | 3 | 1 | 216 |

1) Mutants with significantly increased binding to anti-β-glucan antibody or Dectin-CRD were categorized into very high (3), high (2), moderate (1) or no (0) increase in staining.
2) Mutants with significantly increased elicitation of TNFα were categorized into very high (3), high (2), or moderate (1) increase in elicitation of TNFα from RAW264.7 macrophages
3) Average TNFα elicitation (expresses as percent of elicitation by wild-type control) over greater than three independent experiments. Standard deviations were all less than 20% of average.

Taking gas1Δ as a representative mutant with an intermediate phenotype, increased binding of the anti-β-glucan antibody to this mutant was found, and this binding was blocked by soluble β-glucan. The gas1Δ mutant also binds Dectin-CRD better and elicits a higher level of TNFα from macrophages. The strong correlation between β-glucan exposure and increased TNFα elicitation suggests that β-glucan masking on the surface of Saccharomyces is a key factor in blocking the immune response to fungi.

Most of the genes identified in this screen for the unmasking of β-glucan from the immune system fit under an umbrella of interconnected gene networks that regulate polarized cell wall remodeling: polarization of the actin cytoskeleton, polarized secretion of proteins and polysaccharides, and polarized endocytosis of unwanted byproducts. In addition, several genes required for mannosylation of cell wall proteins (MNN10, MNN11, OCH1, OST3, and OST4) are also required for masking of β-glucan and immune recognition. The connection between mannoprotein processing and β-glucan masking buttresses the idea that a dense coat of mannosylated cell wall proteins masks β-glucan from recognition. Surprisingly, the increase in exposure is due to uncoating or disorganization of the cell wall rather than bulk changes in β-glucan levels, because in those hypereliciting mutants for which the bulk level of β-glucan is known, the vast majority of mutants (14 of 18) has unchanged or lower levels of both types of β-glucan.

As well as finding genes known to play a role in cell wall architecture this screen identified many new genes that likely play roles in cell wall function. Four genes were identified that encode global transcriptional regulatory proteins (ASF1, IES6, MOT2, and SSN8) and six genes with previously unassigned biological function (DIA2, VAC14, RDS2, YMR315W, YNL045W, and YPL158C) were identified that were not expected to be required for masking β-glucan or for recognition by the immune system. The stringency of the screen implicates each of these genes in the cell wall remodeling process.

Some of the mutants with increased binding to anti-β-glucan antibody did not hyperelicit TNFα from macrophages. This could be due to different epitope specificity between antibody and the full-length receptor on the macrophage. To address this issue, all of the “unmasked” mutants were probed with a Dectin-CRD-anti-Myc probe (CRD-Myc) that colocalizes with Dectin-CRD and Dectin-1. This CRD-Myc probe should have a similar size to anti-β-glucan and the same binding specificity as the Dectin-1 receptor. Only three of the 28-non-hyperelicitors showed even marginally increased binding to both Dectin-CRD and CRD-Myc (gup1Δ, kre11Δ, and y1r111wΔ), suggesting that differences in epitope recognition between the anti-β-glucan antibody and receptor account for a majority of the mutants that have greater anti-β-glucan binding but no hyperelicitation.

To examine the surface mannoprotein structure, the level of exposed mannose on the surface of live yeast was quantified by incubating with the mannose-specific lectin concanavalin A (ConA). Although two mutants with known roles in mannosylation (van1Δ, mnn2Δ) showed reduced binding to ConA, a preponderance of mutants with reduced binding was not found among the set of exposed mutants and, importantly, no correlation between ConA binding and TNFα elicitation was found. Therefore, under these conditions, overall levels of mannan on the surface do not appear to regulate TNFα elicitation independently of β-glucan recognition.

The Genetic Pathway for β-Glucan Masking is Conserved in C. albicans

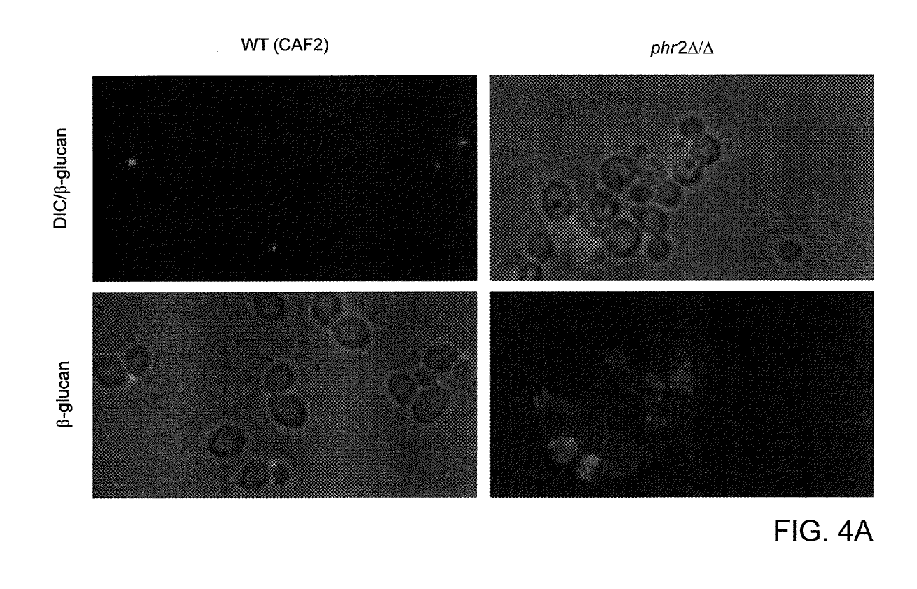

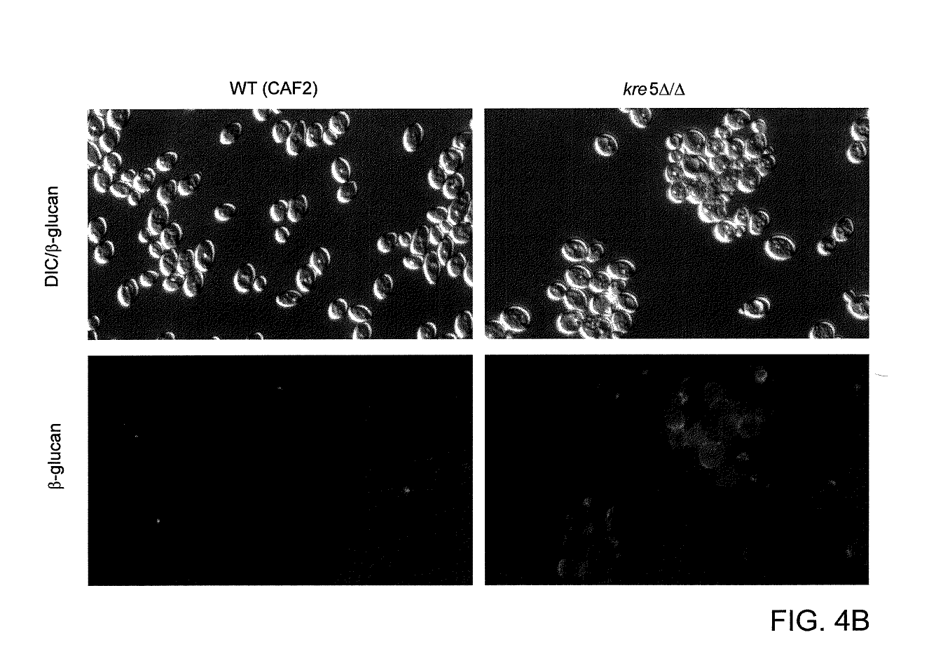

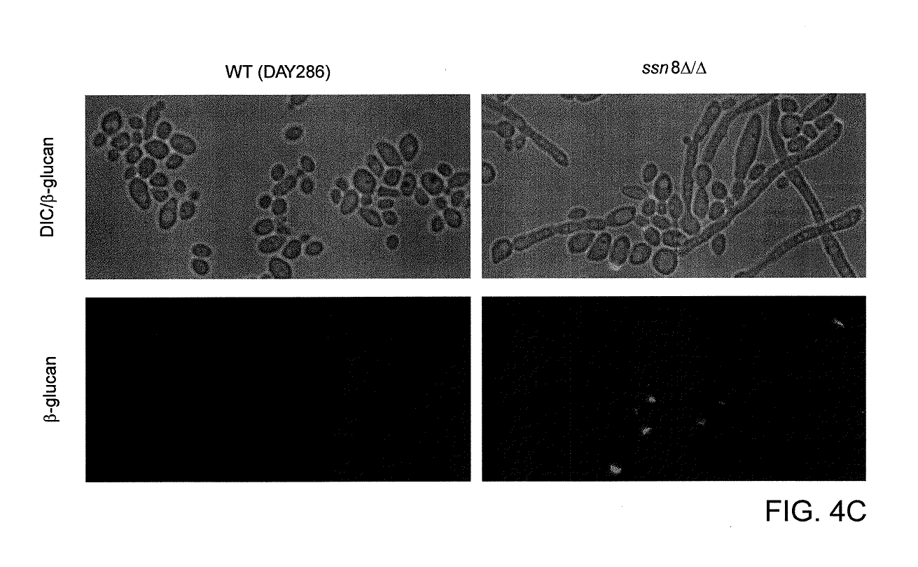

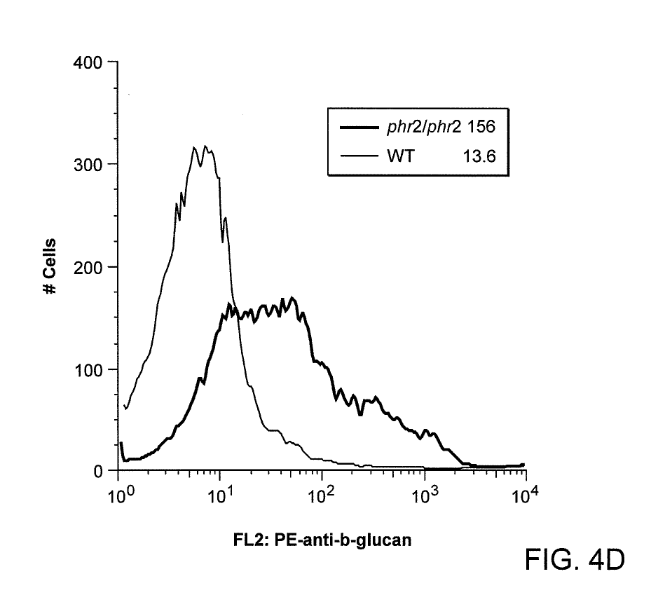

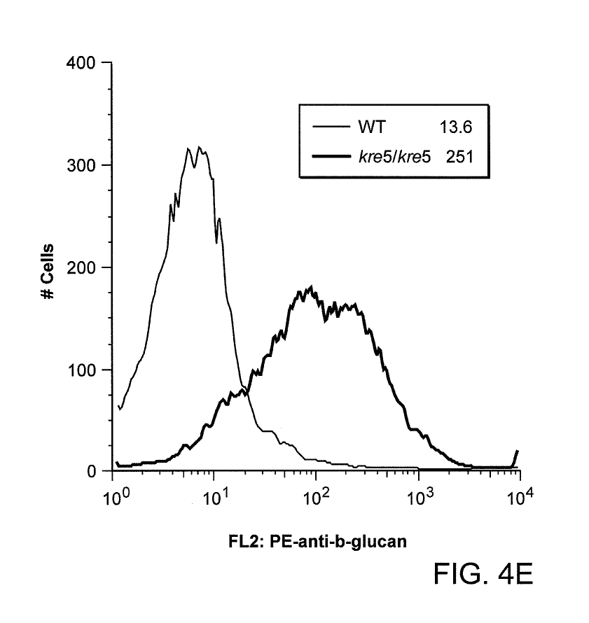

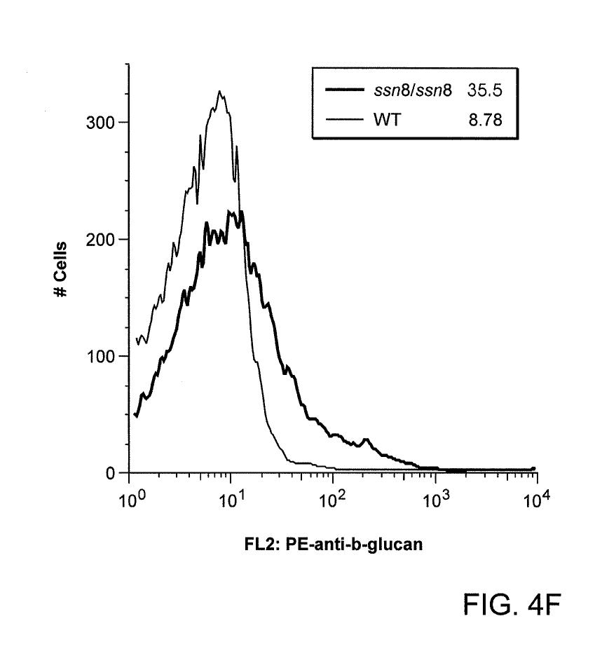

The screen in S. cerevisiae identified potential genes and pathways regulating β-glucan masking in pathogenic fungi. C. albicans homologs might have a similar masking function. Mutations in two of these homologs (PHR2 and KRE5) result in attenuated virulence in mice. Mutations in each of three different β-glucan masking genes caused increased β-glucan exposure in C. albicans (FIG. 4).

FIG. 4 shows that C. albicans mutants of masking genes have more exposed β-Glucan. Wild-type or mutant C. albicans strains were grown overnight in YPD, then stained with anti-β-glucan antibody and Cy3-labeled (FIG. 4 A-C) or PE-labeled (FIG. 4 D-F) secondary antibody. In FIG. 4A-C, the upper photomicrographs show overlay of brightfield and anti-β-glucan staining of Cy3-labeled cells; lower photomicrographs show anti-β-glucan staining alone. FIG. 4D-F show overlay histograms of FACS analysis of PE-labeled cells; data on 20,000 cells are shown. MFI values for wild-type and mutants are shown in insets. In parallel experiments, strains that were complemented with a wild-type copy of the gene showed full reversal of β-glucan exposure (for KRE5) or partial reduction in exposure (for PHR2). This correlates with other phenotypes observed for these complemented strains (De Bernardis F, et al. (1998), Infect Immun 66:3317; Herrero A B, et al., (2004), Eukaryot Cell 3:142320; and Muhlschlegel F A and Fonzi W A (1997), Mol Cell Biol 17:596035).

The PHR2 gene is the only C. albicans homolog of the S. cerevisiae GAS1 gene active under the growth conditions described herein. It encodes a β-glucan transglycosylase required for β-glucan branching, cell wall integrity, and cell wall maintenance. When grown as described above, the phr2Δ/Δ mutant displays a strong increase in the exposure of β-glucan (FIGS. 4A and D).

The homozygous kre5Δ/Δ mutant of C. albicans also showed a dramatic increase in β-glucan exposure (FIGS. 4B and E). Its function in C. albicans was examined because it is nonessential in this fungus yet has a similar function to KRE6, which was identified in the S. cerevisiae screen (the S. cerevisiae kre5Δ mutant is lethal and therefore is not in the deletion library). Both KRE5 and KRE6 play important roles in the synthesis of β1,6-glucan, which is a minor component of the cell wall by weight but is important for cell wall organization and for anchoring of mannoproteins in the wall. In C. albicans, homozygous kre5Δ/Δ mutants are avirulent in the mouse model of infection and have defects in filamentous growth and adherence to epithelial cells (Herrero, et al., 2004).

C. albicans has a single homolog of the S. cerevisiae SSN8 gene, which encodes a global transcriptional regulator. The homozygous ssn8Δ/Δ mutant displays a mild increase in β-glucan exposure that is reproducibly found constrained to the tips of cells and filaments (FIG. 4). This polarized exposure of β-glucan is identical to that found in the phenotype of the S. cerevisiae ssn8Δ mutant, which is also altered in filamentation. The increased exposure of β-glucan at the tips and junctions, which are sites of cell wall remodeling, suggests that in wild-type growth there must be genes such as SSN8 that direct the reconstitution of the wall. The homozygous C. albicans ssn8Δ/Δ mutant is mildly filamentous even in rich (yeast peptone dextrose [YPD]) media, which normally promotes yeast-form growth, showing that filamentation and β-glucan masking appear to be separable phenomena.

Unmasked C. albicans Mutants Elicit More Pro-inflammatory Cytokines through the β-Glucan Receptor

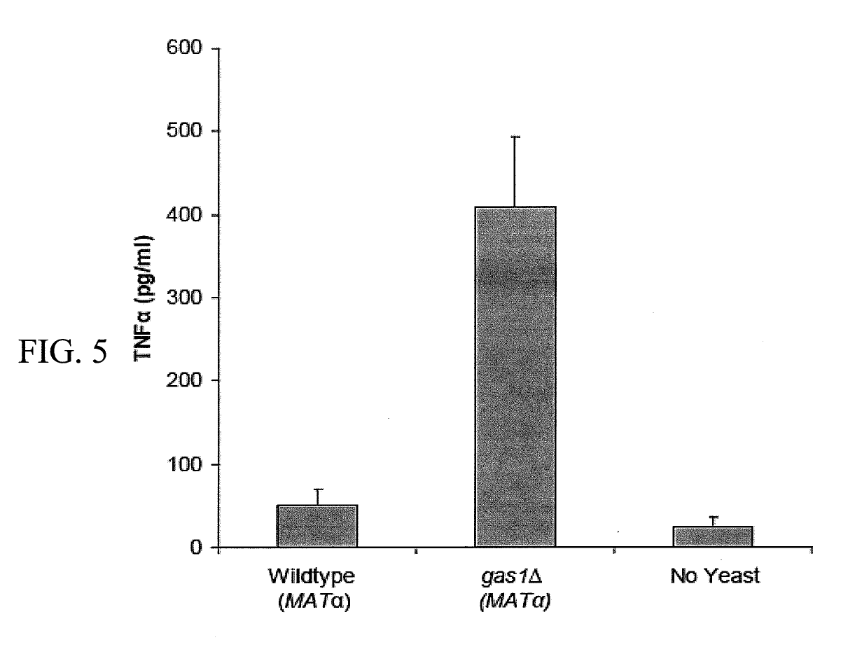

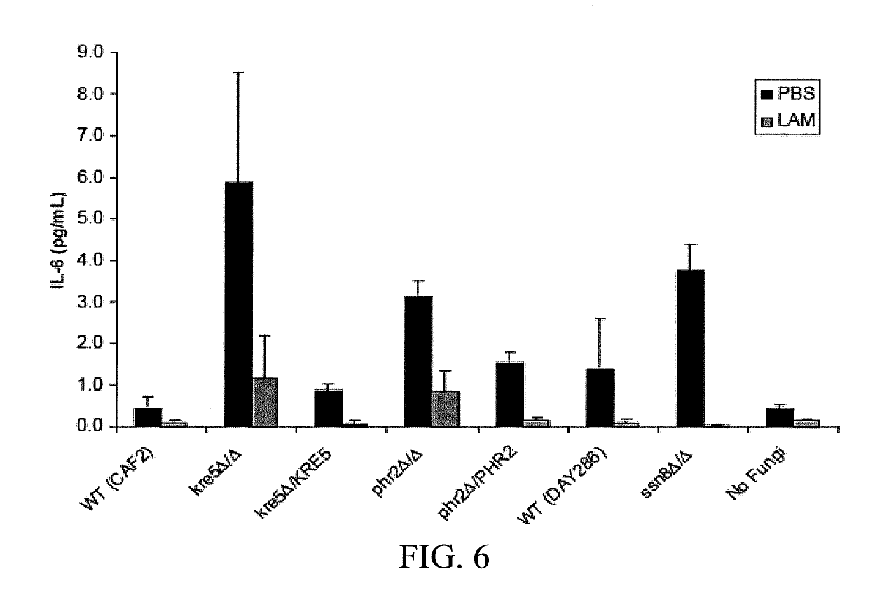

To test whether increased exposure of Candida β-glucan in these mutants also leads to altered immune recognition, wild-type or mutant Candida were exposed to RAW 264.7 macrophages and examined the elicitation of TNFα. As shown in FIG. 1A, the Candida mutants with increased β-glucan exposure elicited higher levels of TNFα from macrophages. The relationship between β-glucan exposure and increased pro-inflammatory response is biologically relevant because unmasked mutants of S. cerevisiae (FIG. 5) and C. albicans (FIG. 1B) also elicited higher levels of TNFα and interleukin 6 (IL-6) (FIG. 6) from BMDMs. This difference in elicitation is not simply a dosage effect, because this phenomenon occurs at different ratios of fungi to macrophages. The altered signaling is also dependent on the β-glucan receptor, because the great majority of the increased elicitation of pro-inflammatory cytokines could be blocked by preincubation of the macrophages with the soluble β-glucan laminarin (FIG. 1C).

FIG. 5 shows TNFα production by BMDMs exposed to different S. cerevisiae strains at a ratio of 5:1 (yeast:macrophage). After fungi were added, macrophages were incubated for 6 h at 37° C., and supernatants were collected for TNFα quantitation.

FIG. 6 shows IL-6 production by BMDMs pretreated for 20 min on ice with medium or soluble β-glucan (laminarin), and then exposed to different C. albicans strains at a ratio of 10:1 (yeast:macrophage). After unbound fungi were washed off, macrophages were incubated for 6 h at 37° C., and supernatants were collected for IL-6 quantitation.

FIG. 1 shows unmasked C. albicans mutants hyperelicit TNFα from macrophages through the β-glucan receptor. In FIG. 1A, C. albicans strains were exposed to RAW264.7 macrophages at a ratio of 2:1 (yeast:macrophage), and supernatants were collected after 6 h. In FIG. 1B, different numbers of C. albicans (wild-type or kre5Δ/Δ mutant) were exposed to BMDMs, and supernatants were collected after 6 h. In FIG. 1C, BMDMs were pretreated for 20 min on ice with media or soluble β-glucan (laminarin); the BMDMs were then exposed to different C. albicans strains at a ratio of 10:1 (yeast:macrophage). After unbound fungi were washed off, macrophages were incubated for 6 h at 37° C., and supernatants were collected for TNFα quantitation.

### Screening

Fungi are grown overnight in YPD rich medium for yeast-form growth and RPMI 1640 for hyphal growth. C. albicans are grown at 37° C.; S. cerevisiae are grown at 30° C. Overnight cultures of C. albicans CAF2 are diluted 1:1,000 into fresh YPD or RPMI 1640 containing dilutions of the candidate compound and grown overnight.

To detect β-glucan exposure, cultures are stained with anti-β-glucan antibody and Cy3-labeled secondary antibody for visualization by microscopy or with PE-labeled secondary antibody for FACS quantification. The results are compared to stained, untreated cultures. Concurrently, cells are labeled briefly with propidium iodide to assess viability and visualized by epifluorescence microscopy or quantified by FACS.

To determine whether the treated cells have an enhanced ability to induce a pro-inflammatory response, cells grown overnight in YPD with or without CF are UV-inactivated and then exposed to BMDMs at a yeast:macrophage ratio of 10:1. Supernatants are taken at 6 h and assayed for TNFα.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A method for determining whether a compound enhances one or more immunogenic properties of a microbial cell, wherein the method comprises the steps of:
a) contacting the compound with a sample comprising at least one microbial cell, and
b) measuring an immunogenic property of the microbial cell, wherein an increase in the immunogenic property relative to an untreated microbial cell indicates that the compound enhances one more immunogenic properties of a microbial cell.

2. The method of claim 1, wherein the compound alters a surface of the cell.

3. The method of claim 1, wherein the immunogenic property is measured by detecting binding of an immune factor to a surface of the microbial cell.

4. The method of claim 3, wherein the immune factor is an antibody.

5. The method of claim 1, wherein the immunogenic property is measured by detecting receptor binding to the microbe.

6. The method of claim 5, wherein the immune receptor is a pattern recognition receptor (PRR).

7. The method of claim 6, wherein the PRR is Dectin-1.

8. The method of claim 1, wherein measuring an immunogenic property comprises determining whether the microbial cell elicits a pro-inflammatory response in vitro or in vivo.

9. The method of claim 8, wherein the pro-inflammatory response is detected by contacting primary macrophages with the microbial cell and measuring of pro-inflammatory cytokine levels produced by the macrophages.

10. The method of claim 9, wherein the pro-inflammatory cytokine is TNFα.

11. The method of claim 9, wherein the pro-inflammatory cytokine is IL-6.

12. The method of claim 1, wherein the microbial cell is a fungal cell.

13. The method of claim 12, wherein the immunogenic property is β-glucan exposure on the surface of the fungal cell.

14. The method of claim 13, wherein measuring the immunogenic property comprises contacting the fungal cell with an anti-β-glucan antibody.

15. The method of claim 13, wherein measuring the immunogenic property comprises contacting the fungal cell with a β-glucan receptor.

16. The method of claim 12, the fungal cell of the sample being selected from the group consisting of Saccharomyces cerevisiae, Candida spp, and Aspergillus fumigatus.

17. The method of claim 1, further comprising prior to step b), inactivating the microbial cell in a manner that the microbial cell remains intact.

18. The method of claim 17, wherein inactivating the microbial cell comprises exposing the microbial cell to ultraviolet light.

19. The method of claim 1, wherein the compound inhibits a gene product of a gene selected from the group consisting of ACE2, ARC18, ARV1, ASF1, BEM1, BEM4, BNI1, CLA4, CNM67, CYK3, DIA2, EDE1, END3, ERD1, FAB1, FEN1, GAS1, GLO3, HEM14, HOF1, IES6, KRE28, KRE6, LAS21, MNN10, MNN11, OCH1, OST3, OST4, PER1, RDS2, PRL16B, RVS161, RVS167, SAC3, SAC6, SEC66, MOT2, SLA1, SLT2, SSN8, TPD3, TUS1, VAC14, VRP1, YMR315W, YNL045W, YPL158C, homologous genes thereof, and orthologous genes thereof.

20. The compound identified by the method of claim 1.

21. A method for determining whether a compound has β-glucan activity, wherein the method comprises the steps of:
a) contacting the compound with a sample comprising at least one fungal cell that contains β-glucan, and
b) determining whether β-glucan can be detected in the sample, wherein detection of the β-glucan indicates that the compound has anti-fungal activity.

22. The method of claim 21, further comprising measuring viability of the fungal cell after step a).

23. The method of claim 21, further comprising prior to step b), inactivating the fungal cell in a manner that the fungal cell remains intact.

24. The method of claim 23, wherein inactivating the fungal cell comprises exposing the fungal cell with ultraviolet light.

25. The method of claim 21, wherein determining whether β-glucan can be detected comprises contacting the fungal cell of the sample with an anti-β-glucan antibody.

26. The method of claim 21, wherein determining whether β-glucan can be detected comprises contacting the fungal cell of the sample with a β-glucan receptor.

27. The method of claim 21, wherein determining whether β-glucan can be detected comprises determining whether the fungal cell elicits a pro-inflammatory response in vitro or in vivo.

28. The method of claim 27, wherein the pro-inflammatory response is detected by contacting primary macrophages with the fungal cell and measuring pro-inflammatory cytokine levels produced by the macrophages.

29. The method of claim 28, wherein the pro-inflammatory cytokine is TNFα.

30. The method of claim 28, wherein the pro-inflammatory cytokine is IL-6.

31. The method of claim 21, the fungal cell of the sample being selected from the group consisting of Saccharomyces cerevisiae, Candida spp., and Aspergillus fumigatus.

32. The method of claim 21, wherein the compound inhibits a gene product of a gene selected from the group consisting of ACE2, ARC18, ARV1, ASF1, BEM1, BEM4, BNI1, CLA4, CNM67, CYK3, DIA2, EDE1, END3, ERD1, FAB1, FEN1, GAS1, GLO3, HEM14, HOF1, IES6, KRE28, KRE6, LAS21, MNN10, MNN11, OCH1, OST3, OST4, PER1, RDS2, PRL16B, RVS161, RVS167, SAC3, SAC6, SEC66, MOT2, SLA1, SLT2, SSN8, TPD3, TUS1, VAC14, VRP1, YMR315W, YNL045W, YPL158C, homologous genes thereof, and orthologous genes thereof.

33. The compound identified by the method of claim 21.

34. A method for determining whether a compound has anti-fungal activity and β-glucan exposing activity comprising:
a) contacting the compound with a first sample comprising at least one fungal cell and determining whether the fungal cell of the sample has been inhibited, and
b) contacting the compound with a second sample comprising at least one fungal cell and determining whether β-glucan can be detected in the sample, wherein inhibition of one or more fungal cells of a) and detection of the β-glucan indicates that the compound has anti-fungal activity and β-glucan exposing activity.

35. The method of claim 34, wherein the second sample is contacted with a sub-inhibitory concentration of the compound.

36. A method for assessing antifungal treatment of a patient comprising measuring β-glucan exposure on fungal cells present in a sample isolated from the patient.

37. The method of claim 36, further comprising treating the sample such that any fungal cells present in the sample are inactivated and in a manner that the fungal cells remain intact.

38. A method for assessing potential antifungal treatment comprising:
a) contacting a first sample obtained from a patient with the candidate compound, the sample comprising at least one fungal cell that contains β-glucan and determining whether the candidate compound inhibits the growth of the fungal cell in vitro, and
b) contacting a second sample obtained from a patient with a candidate compound, the sample comprising at least one fungal cell that contains β-glucan and determining whether β-glucan can be detected in the sample, wherein detection of the β-glucan indicates that the compound is useful as an antifungal treatment.

39. The method of claim 38, wherein the candidate compound fails to inhibit the growth of the fungal cell in vitro.

40. A method of treating fungal infection in an individual comprising administering the compound identified by the method of claim 39.

41. A kit for determining whether a compound causes the exposure of β-glucan on a fungal cell comprising:
a) means for detecting β-glucan on fungal cells and
b) means for measuring levels of pro-inflammatory response caused by the fungal cell.
